(19) United States
(12) Patent Application Publication
(10) Pub. No.: US 2024/0164875 A1
(43) Pub. Date: May 23, 2024

(54) METHOD AND SYSTEM FOR PRESENTING DENTAL SCAN

(71) Applicant: GET-GRIN INC., Airmont, NY (US)

(72) Inventors: Carmi RAZ, Gizo (IL); Pamela Sharon OREN-ARTZI, Airmont, NY (US); Yarden EILAT-BLOCH, Haifa (IL)

(21) Appl. No.: 18/424,237

(22) Filed: Jan. 26, 2024

Related U.S. Application Data
(63) Continuation of application No. PCT/US2022/038737, filed on Jul. 28, 2022.
(60) Provisional application No. 63/227,076, filed on Jul. 29, 2021.

(51) Int. Cl. A61C 9/00; A61C 7/00; G06T 7/00; G16H 30/40

(52) U.S. Cl. CPC A61C 9/0053; A61C 7/002; G06T 7/00; G16H 30/40; G06T 2207/10016; G06T 2207/30036

(57) ABSTRACT

In an aspect, the present disclosure provides a method to generate a dental scan clip, comprising: (a) providing a clip start point, a clip end point, and a dental scan parameter; and (b) editing a dental scan in accordance with the clip start point, the clip end point, and the dental scan parameter to generate the dental scan clip.

### CROSS-REFERENCE TO RELATED APPLICATIONS

This application is a continuation of International Patent Application PCT/US22/38737 filed Jul. 28, 2022, which claims the benefit of U.S. Provisional Application Ser. No. 63/227,076 filed Jul. 29, 2021, each of which is incorporated by reference herein in its entirety.

### TECHNICAL FIELD

The present invention, in at least some embodiments, relates to a method and a system for presenting dental imaging, and more specifically a method and system for editing and enabling comparison of dental scans.

### BACKGROUND

Dental professionals and orthodontists may treat and monitor a patient's dental condition based on in-person visits. Treatment and monitoring of a patient's dental condition may require a patient to schedule multiple in-person visits to a dentist or orthodontist. The quality of treatment and the accuracy of monitoring may vary depending on how often and how consistently a patient sees a dentist or orthodontist. In some cases, suboptimal treatment outcomes may result if a patient is unable or unwilling to schedule regular visits to a dentist or orthodontist.

### SUMMARY

Recognized herein is a need for remote dental monitoring solutions to allow dental patients to receive high quality dental care, without requiring a dental professional to be physically present with the patient. Some dental professionals and orthodontists may use conventional teledentistry solutions to accommodate patients' needs and schedules. However, such conventional teledentistry solutions may provide inadequate levels of supervision. Further, such conventional teledentistry solutions may be limited by an inaccurate or insufficient monitoring of a patient's dental condition based on one or more photos taken by the patient, if the photos do not adequately capture various intraoral features.

The present disclosure provides a method for editing a dental scan obtained through intraoral imaging. As used herein, intraoral imaging may refer to the acquisition of one or more intraoral videos and/or intraoral images. The methods and systems disclosed herein may provide a convenient solution and user experience for dental patients to capture one or more intraoral videos or images using a mobile device such as a smartphone. The methods and systems disclosed herein may provide dentists and orthodontists with a detailed analysis of the patient's dental condition based on one or more full arch scans captured remotely by the patient.

In an aspect, the present disclosure provides a method to generate a dental scan clip, comprising: (a) providing a clip start point, a clip end point, and a dental scan parameter; and (b) editing a dental scan in accordance with the clip start point, the clip end point, and the dental scan parameter to generate the dental scan clip.

In some cases, the clip start point is determined by a point in the dental scan at which a dental landmark is identified within the dental scan. In some cases, the dental landmark is a maxillary tuberosity. In some cases, the dental landmark is a retromolar pad. In some cases, the dental landmark is a tooth. In some cases, the clip end point is determined by a point in the dental scan at which a dental landmark is no visible in the dental scan. In some cases, the dental landmark is a maxillary tuberosity. In some cases, the dental landmark is a retromolar pad. In some cases, the dental landmark is a tooth. In some cases, the clip end point is determined by a point in the dental scan at which a dental landmark is identified within the dental scan. In some cases, the dental landmark is a maxillary tuberosity. In some cases, the dental landmark is a retromolar pad. In some cases, the dental landmark is a tooth. In some cases, the clip start point is at a time point outside of the dental scan. In some cases, the clip end point is at a time point outside of the dental scan. In some cases, the clip start point is determined by a point in the dental scan where a first maxillary tuberosity is identified within the dental scan and the clip end point is determined by a point in the dental scan where a second maxillary tuberosity is no longer visible within the dental scan. In some cases, the clip start point is determined by a point in the dental scan where a first tooth is identified within the dental scan and the clip end point is determined by a point in the dental scan where a second tooth is no longer visible within the dental scan.

In some cases, the dental scan parameter comprises ensuring an upper teeth arch remains horizontal in the dental scan frame. In some cases, the dental scan parameter comprises ensuring a lower teeth arch remains horizontal in the dental scan frame. In some cases, the dental scan parameter comprises ensuring one or more spaces between two or more teeth remains vertical in the dental scan frame. In some cases, the dental scan parameter comprises ensuring an imaginary line between two dental landmarks remains horizontal in the dental scan frame.

In some cases, the dental scan parameter comprises ensuring an imaginary line parallel to a bite arch remains horizontal in the dental scan frame. In some cases, the dental scan parameter comprises ensuring the movement speed of a dental scan remains constant. In some cases, the dental scan provided is comprises one or more videos or images. In some cases, the dental scan provided is a video or image acquired with a mobile device. In some cases, the editing comprises changing the scale of at least part of the provided dental scan to match the scale of a reference dental scan clip. In some cases, the editing comprises rotating at least part of the provided dental scan to match the orientation of a reference dental scan clip. In some cases, the editing comprises changing the frames per second displayed of at least part of the provided dental scan to match a movement speed of a reference dental scan clip. In some cases, the editing comprises aligning the display of at least part of the provided dental scan with a reference dental scan clip. In some cases, the editing comprises changing the display perspective of at least part of the provided dental scan to match the display perspective of a reference dental scan clip. In some cases, the editing comprises adding transparent or colorful effects to at least part of the provided dental scan.

In some cases, the dental scan clip is a movie, video, or animation. In some cases, the dental scan clip is less than 1 minute long. In some cases, the method further comprises presenting the dental scan clip simultaneously with a reference dental scan clip. In some cases, the method further comprises underlying or overlying the dental scan clip with a reference dental scan clip. In some cases, differences between the dental scan clip and the reference dental scan clip are marked. In some cases, the dental scan clip and reference dental scan clip are obtained from a subject. In some cases, the dental scan clip is obtained from the subject at a later date than the reference dental scan clip.

In another aspect, the present disclosure provides a method to transform three dimensional (3D) dental data into a dental model clip, comprising: (a) providing the 3D dental data; (b) providing a graphic visualization tool; (c) using the graphic visualization tool to generate a dental model from the 3D dental data; (d) providing a clip start point, a clip end point, and a dental model parameter; and (e) generating the dental model clip in accordance with the clip start point, the clip end point, and the dental model parameter.

In some cases, the clip start point is determined by a point in the dental model at which a dental landmark is identified within the dental model. In some cases, the dental landmark is a maxillary tuberosity. In some cases, the dental landmark is a retromolar pad. In some cases, the dental landmark is a tooth. In some cases, the clip end point is determined by a point in the dental model at which a dental landmark is no visible in the dental model. In some cases, the dental landmark is a maxillary tuberosity. In some cases, the dental landmark is a retromolar pad. In some cases, the dental landmark is a tooth. In some cases, the clip end point is determined by a point in the dental model at which a dental landmark is identified within the dental model. In some cases, the dental landmark is a maxillary tuberosity. In some cases, the dental landmark is a retromolar pad. In some cases, the dental landmark is a tooth. In some cases, the clip start point is determined by a point in the dental model where a first maxillary tuberosity is identified within the dental model and the clip end point is determined by a point in the dental model where a second maxillary tuberosity is no longer visible within the dental model. In some cases, the clip start point is determined by a point in the dental model where a first tooth is identified within the dental model and the clip end point is determined by a point in the dental model where a second tooth is no longer visible within the dental model. In some cases, the clip start point is at a time point outside of the dental model. In some cases, the clip end point is at a time point outside of the dental model.

In some cases, the dental model parameter comprises ensuring an upper teeth arch remains horizontal in the dental model frame. In some cases, the dental model parameter comprises ensuring a lower teeth arch remains horizontal in the dental model frame. In some cases, the dental model parameter comprises ensuring one or more spaces between two or more teeth remains vertical in the dental model frame. In some cases, the dental model parameter comprises ensuring an imaginary line between two dental landmarks remains horizontal in the dental model frame. In some cases, the dental model parameter comprises ensuring an imaginary line parallel to a bite arch remains horizontal in the dental model frame. In some cases, the dental model parameter comprises ensuring the movement speed of a dental model remains constant.

In some cases, the dental model comprises one or more videos or images. In some cases, the dental model is a video or image acquired with a mobile device. In some cases, the generating comprises changing the scale of at least part of the provided dental model to match the scale of a reference dental model clip. In some cases, the generating comprises rotating at least part of the provided dental model to match the orientation of a reference dental model clip. In some cases, the generating comprises changing the frames per second displayed of at least part of the provided dental model to match a movement speed of a reference dental model clip. In some cases, the generating comprises aligning the display of at least part of the provided dental model with a reference dental model clip. In some cases, the generating comprises changing the display perspective of at least part of the provided dental model to match the display perspective of a reference dental model clip. In some cases, the generating comprises adding transparent or colorful effects to at least part of the provided dental model.

In some cases, the dental model clip is a movie, video, or animation. In some cases, the dental model clip is less than 1 minute long. In some cases, the method further comprises presenting the dental model clip simultaneously with a reference dental scan clip. In some cases, the method further comprises underlying or overlying the dental model clip with a reference dental scan clip. In some cases, differences between the dental model clip and the reference dental scan clip are marked.

In another aspect, the present disclosure provides a computer-implemented method for remote dental monitoring, the method comprising: providing a dental scan clip of a user; providing a reference dental scan clip of the user or a dental model clip of the user; and simultaneously presenting the dental scan clip with the reference dental scan clip or the dental model clip on a graphical display.

As used herein, the term “dental scan” refers to a video or an image capture of the intraoral perspective imaging of the teeth arch or of a tooth.

Unless otherwise defined, all technical and scientific terms used herein have the same meaning as commonly understood by one of ordinary skill in the art regarding the devices, systems, and methods described herein. Although suitable methods and materials are described below, methods and materials similar or equivalent to those described herein can be used in the practice of the systems and methods described herein. In case of conflict, the patent specification, including definitions, will control. All materials, methods, and examples are illustrative only and are not intended to be limiting.

As used herein, the terms “comprising” and “including” or grammatical variants thereof are to be taken as specifying inclusion of the stated features, integers, actions or components without precluding the addition of one or more additional features, integers, actions, components or groups thereof. This term is broader than, and includes the terms “consisting of” and “consisting essentially of” as defined by the Manual of Patent Examination Procedure of the United States Patent and Trademark Office.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or readily developed from known manners, means, techniques and procedures by practitioners of architecture and/or computer science.

Implementation of the methods and systems described herein involves performing or completing selected tasks or steps manually, automatically, or a combination thereof. Moreover, according to actual instrumentation and equipment of embodiments of methods, apparatus and systems described herein, several selected steps could be implemented by hardware or by software on any operating system of any firmware or a combination thereof. For example, as hardware, selected steps could be implemented as a chip or a circuit. As software, selected steps could be implemented as a plurality of software instructions being executed by a computer using any suitable operating system. In any case, selected steps of the methods and systems described herein could be described as being performed by a data processor, such as a computing platform for executing a plurality of instructions.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

In order to understand the systems and methods described herein and to see how it may be carried out in practice, embodiments will now be described, by way of non-limiting examples only, with reference to the accompanying figures. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIG. 1 is a flow-chart schematically illustrating a method for generating a dental scan clip, in accordance with some embodiments.

FIG. 2 is a flow-chart schematically illustrating a method for transforming three dimension (3D) dental data into a dental model clip, in accordance with some embodiments.

FIG. 3 is a flow-chart schematically illustrating a method for remote dental monitoring, in accordance with some embodiments.

FIG. 4 schematically illustrates a computer system that is programmed or otherwise configured to implement at least some of the methods disclosed herein, in accordance some embodiments.

### DETAILED DESCRIPTION

While various embodiments have been shown and described herein, it will be obvious to those skilled in the art that such embodiments are provided by way of example only. Numerous variations, changes, and substitutions may occur to those skilled in the art without departing from the methods and systems described herein. It should be understood that various alternatives to the embodiments of the systems and methods described herein may be employed.

The term “real-time,” as used herein, generally refers to a simultaneous or substantially simultaneous occurrence of a first event or action with respect to an occurrence of a second event or action. A real-time action or event may be performed within a response time of less than one or more of the following: ten seconds, five seconds, one second, a tenth of a second, a hundredth of a second, a millisecond, or less relative to at least another event or action. A real-time action may be performed by one or more computer processors.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “a,” “an,” and “the,” as used herein, generally refer to singular and plural references unless the context clearly dictates otherwise.

### Overview

According to an aspect, the present disclosure provides methods and systems for intraoral assessment. As used herein, “intraoral assessment” may refer to an assessment of an appearance or condition of an intraoral region of a subject. The methods and systems disclosed herein can transfer one or more intraoral videos or images taken by users using a mobile device, such as a smartphone, into a dental scan clip. The methods and systems disclosed herein may provide dentists and orthodontists with a detailed analysis of the patient's dental condition based on a video or on one or more images of at least one tooth, such as one or more teeth, one or more dental arches or both dental arches of a subject. In some cases, the videos or one or more images are captured remotely by a user. The videos or images may be converted by the methods described herein into a dental scan clip. In some embodiments, the user is the subject of the intraoral imaging. In some embodiments the user is a person other than the subject. In some cases, the user is present at the same location as the subject of the intraoral imaging and assessment of the captured video or image(s) is performed at a remote location.

As used herein the term “dental scope” or “dental adaptor” refer to a device that can be attached to a camera or mobile phone and enables capturing at least one intraoral image or videos of a subject. An example and possible uses can be seen in U.S. patent application Ser. No. 17/336,997. The methods and systems of the present disclosure may be implemented using a software application that is configured to enable a dental patient to capture videos and/or images of intraoral regions. The software application may be used by a user or a subject (e.g., a dental patient) in conjunction with a mobile device to remotely monitor a dental condition of the subject. The dental condition may comprise a development, a growth, a movement, an appearance, a condition, a physical arrangement, a position, and/or an orientation of the subject's teeth or/and the subject's soft tissues in the oral cavity.

The intraoral adaptor may be used to create a dental scan that can be converted, according to the present disclosure, to a dental scan clip. This dental scan clip can be used in remote monitoring. As used herein, “remote monitoring” may refer to monitoring a condition of an intraoral region of a subject, wherein the monitoring is performed at one or more locations remote from the subject. For example, a dentist or a medical specialist may monitor the intraoral anatomy or intraoral condition at a first location that is different than a second location at which the subject is located. The first location and the second location may be separated by a distance spanning at least 1 meter, 1 kilometer, 10 kilometers, 100 kilometers, 1000 kilometers, or more.

The remote monitoring may be performed by assessing an intraoral condition of the subject using one or more dental scan clips generated from one or more dental scans captured from the subject when the subject is located remotely from the dentist or a dental office. In some cases, the remote monitoring may be performed in real-time such that a dentist or other healthcare provider is able to assess the dental condition when a subject uses a mobile device to acquire one or more videos or images of one or more intraoral regions in the patient's mouth. The remote monitoring may be performed using equipment, hardware, and/or software that is not physically located at a dental office.

As used herein the term “dental scan clip” refers to a movie, video or animation created from a dental scan of a subject. The dental scan may comprise intraoral images or videos. In some cases, the dental scan clip has been edited from a full dental scan. In some cases, the dental scan clip includes videos or images of the teeth of the user. The videos or images of the teeth of the user may be captured using a capturing device such as a mobile device or smartphone. The dental scan clip may be created by selection of a start point and end point from a full-length dental scan.

As used herein the term “dental model clip” refers to a movie, video or animation generated from a 3D model of an intraoral region. The 3D model may represent the intraoral region of a real patient or user. The 3D model may represent a generic 3D model of an intraoral region that is not associated with the user. For example, the generic 3D model may be based off of a baseline or idealized intraoral region from a training model. The dental model may comprise intraoral images or videos. In some cases, the dental model clip has been generated generated from the 3D model of an intraoral region. In some cases, the dental model clip includes videos or images of the 3D model teeth. The dental model clip may be created by selection of a start point and end point from a 3D model of an intraoral region.

As used herein the term “clip starting point” refers to a specific location where the dental scan clip or dental model clip begins. In some cases, the clip starting point is triggered when a certain anatomical feature is located in a dental scan of the user's mouth or in a 3D model of an intraoral region.

As used herein the term “clip end point” refers to a specific location in a dental scan where the dental scan clip or dental model clip ends. In some cases, the clip end point is triggered when a certain anatomical feature or dental landmark can no longer be seen in the user's mouth or in a 3D model of an intraoral region.

As used herein the term “3D dental data” refers to a file or computer data that represents the appearance of the intraoral region of a user or generic intraoral imaging.

As used herein the term “3D model” refers to a file or computer data that represents the appearance of the intraoral region of a a user or generic intraoral imaging that can be presented as a copy of user or a generic intraoral copy.

As used herein, the term “graphic visualization tool” refers to any tool or device used to generate a dental model from 3D dental data. The graphic visualization tool may comprise a presentation tool which can be used to present and move the 3D dental data as a 3D model. Data points (perspective acquiring points) correlating to the orientation, scale, and/or angle of the 3D dental data may be used to create a dental model.

### Dental Scan Clip

In an aspect, the present disclosure provides a method to generate a dental scan clip, comprising: Providing clip start point, clip end point and a parameter to keep; Providing a dental scan; Editing the dental scan to in accordance to the clip start point, the clip end point while keeping the parameter.

In some embodiments the clip start point, the clip end point determine as the point that the maxillary tuberosity or the retromolar pad is identified.

In some preferred embodiments the maxillary tuberosity or the retromolar pad are visible.

In some embodiments the clip start point, the clip end point determine as the location of two teeth.

In some embodiments the parameter is one of; upper teeth arch horizontal in the frame, lower teeth arch horizontal in the frame, spaces between teeth vertical in the frame, imaginary line of teeth arches bite horizontal in the frame, objects movement speed is constant.

In some embodiments the dental scan provided is one or more movie or images.

In some preferred embodiments the dental scan provided is a video or image acquire with mobile device.

In some embodiments the editing comprises changing scale of at least part of provided dental scan.

In some embodiments the editing comprises rotation of at least part of provided dental scan.

In some embodiments the editing comprises changing the frames per second display of at least part of provided dental scan.

In some embodiments the editing comprises alignment the display of at least part of provided dental scan.

In some embodiments the editing comprises changing the display perspective of at least part of provided dental scan.

In some embodiments the editing comprises adding transparent or color affects display of at least part of provided dental scan.

In some embodiments the dental scan clip is a movie, video, or animation.

In some embodiments the dental scan clip is less than 2 minute long, 1.5 minute long, 1 minute long, 0:45 minute long, 0:30 minute long,

In some embodiments the dental scan clip is more than 0.15 minute long, 0.30 minute long, 0.45 minute long, 1 minute long.

In some embodiments the present disclosure further comprising presenting the dental scan clip simultaneously with a prior generated dental scan clip.

In some embodiments the present disclosure further comprising presenting the dental scan clip overlaying or underlaying with a prior generated dental scan clip.

In some embodiments the differences between the dental scan clip and prior generated dental scan clip are marked.

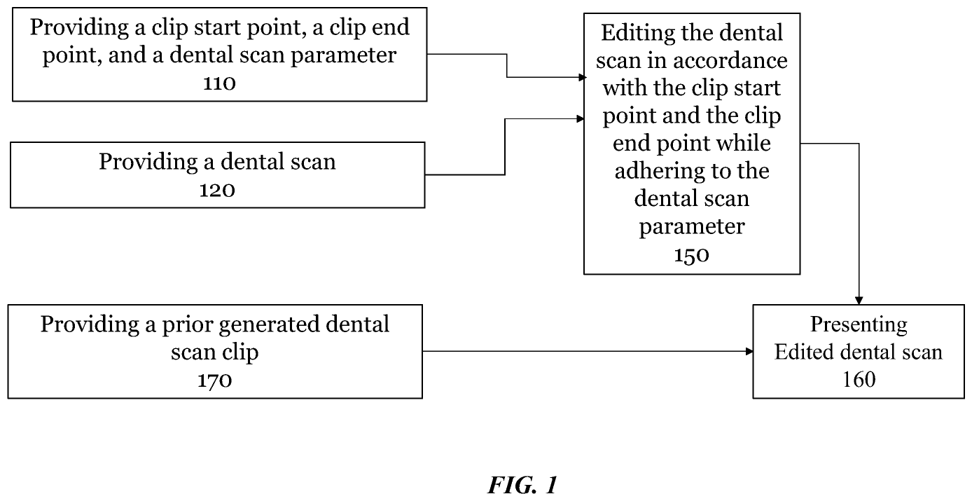

FIG. 1 is a flow-chart schematically illustrating a method for generating a dental scan clip, in accordance with some embodiments. As shown, the method may include providing a clip start point, a clip end point, and a dental scan parameter 110; providing a dental scan 120; editing the dental scan in accordance to the clip start point and the clip end point while adhering to the dental scan parameter 150 during the dental scan; and presenting the edited dental scan 160. In some embodiments, presenting the edited dental scan can comprise presenting a prior generated dental scan 170.

In an aspect, the present disclosure provides a system to generate a dental scan clip, in accordance with the present disclosure.

In some embodiments, the dental scan clip can be generated in a mobile device, a remote server, or in other computer system. In an aspect, the present disclosure provides a method to generate a dental scan clip, comprising: (a) providing a clip start point, a clip end point, and a dental scan parameter; (b) providing a dental scan; and (c) editing the dental scan in accordance with the clip start point, the clip end point, and the dental scan parameter.

In some cases, the clip start point is determined by a point in the dental scan at which a dental landmark is identified within the dental scan. In some cases, the dental landmark is a maxillary tuberosity. In some cases, the dental landmark is a retromolar pad. In some cases, the dental landmark is a tooth.

In some cases, the clip end point is determined by a point in the dental scan at which a dental landmark is no visible in the dental scan. In some cases, the dental landmark is a maxillary tuberosity. In some cases, the dental landmark is a retromolar pad. In some cases, the dental landmark is a tooth.

In some cases, the clip end point is determined by a point in the dental scan at which a dental landmark is identified within the dental scan. In some cases, the dental landmark is a maxillary tuberosity. In some cases, the dental landmark is a retromolar pad. In some cases, the dental landmark is a tooth.

In some cases, the clip start point is determined by a point in the dental scan where a first maxillary tuberosity is identified within the dental scan and the clip end point is determined by a point in the dental scan where a second maxillary tuberosity is no longer visible within the dental scan.

In some cases, the clip start point is determined by a point in the dental scan where a first tooth is identified within the dental scan and the clip end point is determined by a point in the dental scan where a second tooth is no longer visible within the dental scan.

In some cases, the clip start point is at a time point outside the window of the dental scan. For example, the clip start point may be at a time point before the dental scan begins. In some cases, the point is imaginary. In some cases, the clip end point is at a time point outside the window of the dental scan. For example, the clip end point may be at a time after the dental scan has ended. In some cases, the point is imaginary.

In some cases, the dental scan parameter comprises ensuring an upper teeth arch remains horizontal in the dental scan frame. In some cases, the dental scan parameter comprises ensuring a lower teeth arch remains horizontal in the dental scan frame. In some cases, the dental scan parameter comprises ensuring one or more spaces between two or more teeth remains vertical in the dental scan frame. In some cases, the dental scan parameter comprises ensuring an imaginary line between two dental landmarks remains horizontal in the dental scan frame. In some cases, the dental scan parameter comprises ensuring an imaginary line parallel to a bite arch remains horizontal in the dental scan frame. In some cases, the dental scan parameter comprises ensuring the movement speed of a dental scan remains constant.

In some cases, the dental scan provided is comprises one or more videos or images. In some cases, the dental scan provided is a video or image acquired with a mobile device.

In some cases, the editing comprises changing the scale of at least part of the provided dental scan to match the scale of a reference dental scan clip. In some cases, the editing comprises rotating at least part of the provided dental scan to match the orientation of a reference dental scan clip. In some cases, the editing comprises changing the frames per second displayed of at least part of the provided dental scan to match the movement speed of a reference dental scan clip. In some cases, the editing comprises aligning the display of at least part of the provided dental scan with a reference dental scan clip. In some cases, the editing comprises changing the display perspective of at least part of the provided dental scan to match the display perspective of a reference dental scan clip.

In some cases, the editing comprises adding transparent or colorful effects to at least part of the provided dental scan. In some cases, the dental scan clip is a movie, video, or animation.

In some cases, the dental scan clip is less than 1 minute long. In some cases, the dental scan clip is about 0.25 minutes, about 0.5 minutes, about 1 minute, about 1.5 minutes, or about 2 minutes. In some cases, the dental scan clip is at least about 0.25 minutes, about 0.5 minutes, about 1 minute, or about 1.5 minutes. In some cases, the dental scan clip is at most about 0.5 minutes, about 1 minute, about 1.5 minutes, or about 2 minutes.

In some cases, the method further comprises presenting the dental scan clip simultaneously with a reference dental scan clip. In some cases, the method further comprises underlying or overlying the dental scan clip with a reference dental scan clip. In some cases, differences between the dental scan clip and the reference dental scan clip are marked. In some cases, the dental scan clip and reference dental scan clip are obtained from one patient. In some cases, the dental scan clip is obtained from the patient at a later date than the reference dental scan clip.

The differences between the dental scan clip and the reference clip may be used to evaluate or monitor the progress of a dental or orthodontia treatment plan for a patient. The dental condition may comprise (i) a movement of one or more teeth of the subject, (ii) an accumulation of plaque on the one or more teeth of the subject, (iii) a change in a color or a structure of the one or more teeth of the subject, (iv) a change in a color or a structure of a tissue adjacent to the one or more teeth of the subject, and/or (v) a presence or lack of presence of one or more cavities. In some cases, the differences between the dental scan clip and the reference clip may be used to assess the subject's actual progress in relation to a dental treatment plan based at least in part on a comparison of (i) the one or more changes in the dental structure or the dental condition of the subject and (ii) a planned or estimated change in the dental structure or the dental condition of the subject.

### Dental Model Clip

In another aspect, the present disclosure provides a method to transform a three dimension (3D) dental data into dental model clip, comprising: Providing a 3D dental data; providing a presentation toll for the 3D dental data; Providing a perspective acquiring points data, Providing clip start point, clip end point and a parameter to keep; generating dental model clip in accordance to the clip start point, the clip end point while keeping the parameter.

In some embodiments the clip start point, the clip end point determine as the point that the maxillary tuberosity or the retromolar pad is identified.

In some preferred embodiments the maxillary tuberosity or the retromolar pad are visible.

In some embodiments the clip start point, the clip end point determine as the location of two teeth.

In some embodiments the parameter is one of; upper teeth arch horizontal in the frame, lower teeth arch horizontal in the frame, spaces between teeth vertical in the frame, imaginary line of teeth arches bite horizontal in the frame, objects movement speed is constant.

In some embodiments the 3D presentation tool provided can present and move the 3D dental data as a 3D model

In some embodiments the perspective acquiring points data provided are used during the present and move the 3D dental data as a 3D model

In some embodiments the generating comprises changing scale of at least part of provided 3D dental data.

In some embodiments the generating comprises rotation of at least part of provided 3D dental data.

In some embodiments the generating comprises changing the display perspective of at least part of provided 3D dental data.

In some embodiments the generating comprises adding transparent or color affects display of at least part of provided 3D dental data.

In some embodiments the dental model clip is a movie, video, or animation.

In some preferred embodiments the dental model clip is less than 2 minute long, 1.5 minute long, 1 minute long, 0:45 minute long, 0:30 minute long,

In some preferred embodiments the dental model clip is more than 0.15 minute long, 0.30 minute long, 0.45 minute long, 1 minute long.

In some embodiments the present disclosure further comprising presenting the dental model clip simultaneously with a dental scan clip.

In some embodiments the present disclosure further comprising presenting the dental model clip overlaying or underlaying with a dental scan clip.

In some embodiments the differences between the dental model clip and dental scan clip are marked.

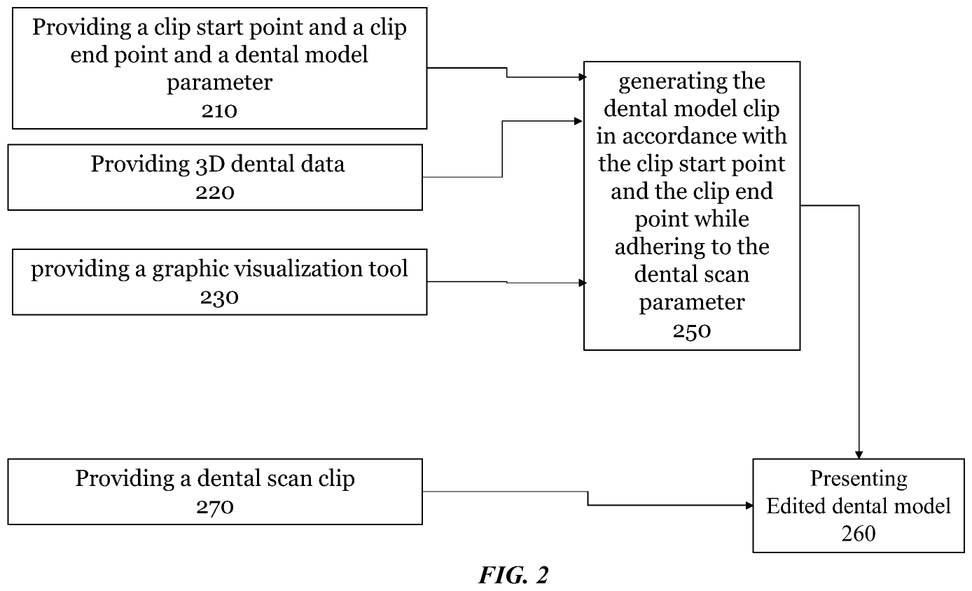

FIG. 2 is a flow-chart schematically illustrating a method for generating a dental model clip, in accordance with some embodiments. As shown, the method may include: providing a clip start point, a clip end point, and a dental model parameter 210; providing 3D dental data 220;

providing a graphic visualization tool 230; generating dental model clip in accordance to the clip start point and the clip end point while adhering to the dental model parameter 250. In some embodiments, presenting the edited dental model 260 can comprise comparing the edited dental model 260 with a dental scan clip 270.

In an aspect, the present disclosure provides a system to generate a dental model clip, in accordance to present disclosure.

In some embodiments, the dental model clip can be generated using a mobile device, a remote server, or another computer system.

In an aspect, the present disclosure provides a method to transform three dimensional (3D) dental data into a dental model clip, comprising: (a) providing the 3D dental data; (b) providing a graphic visualization tool; (c) using the graphic visualization tool to generate a dental model from the 3D dental data; (d) providing a clip start point, a clip end point, and a dental model parameter; and (e) generating the dental model clip in accordance with the clip start point, the clip end point, and the dental model parameter.

The 3D dental data may be a 3D surface model (mesh) with fine details of the surface of the dental structure

In some cases, the clip start point is determined by a point in the dental model at which a dental landmark is identified within the dental model. In some cases, the dental landmark is a maxillary tuberosity. In some cases, the dental landmark is a retromolar pad. In some cases, the dental landmark is a tooth.

In some cases, the clip end point is determined by a point in the dental model at which a dental landmark is no visible in the dental model. In some cases, the dental landmark is a maxillary tuberosity. In some cases, the dental landmark is a retromolar pad. In some cases, the dental landmark is a tooth.

In some cases, the clip end point is determined by a point in the dental model at which a dental landmark is identified within the dental model. In some cases, the dental landmark is a maxillary tuberosity. In some cases, the dental landmark is a retromolar pad. In some cases, the dental landmark is a tooth.

In some cases, the clip start point is at a time point outside the window of the dental model. For example, the clip start point may be at a time point before the dental model begins. In some cases, the point is imaginary. In some cases, the clip end point is at a time point outside the window of the dental model. For example, the clip end point may be at a time after the dental model has ended. In some cases, the point is imaginary.

In some cases, the clip start point is determined by a point in the dental model where a first maxillary tuberosity is identified within the dental model and the clip end point is determined by a point in the dental model where a second maxillary tuberosity is no longer visible within the dental model.

In some cases, the clip start point is determined by a point in the dental model where a first tooth is identified within the dental model and the clip end point is determined by a point in the dental model where a second tooth is no longer visible within the dental model.

In some cases, the dental model parameter comprises ensuring an upper teeth arch remains horizontal in the dental model frame. In some cases, the dental model parameter comprises ensuring a lower teeth arch remains horizontal in the dental model frame. In some cases, the dental model parameter comprises ensuring one or more spaces between two or more teeth remains vertical in the dental model frame. In some cases, the dental model parameter comprises ensuring an imaginary line between two dental landmarks remains horizontal in the dental model frame. In some cases, the dental model parameter comprises ensuring an imaginary line parallel to a bite arch remains horizontal in the dental model frame. In some cases, the dental model parameter comprises ensuring the movement speed of a dental model remains constant.

In some cases, the dental model comprises one or more videos or images. In some cases, the dental model is a video or image acquired with a mobile device.

In some cases, the generating comprises changing the scale of at least part of the provided dental model to match the scale of a reference dental model clip. In some cases, the generating comprises rotating at least part of the provided dental model to match the orientation of a reference dental model clip. In some cases, the generating comprises changing the frames per second displayed of at least part of the provided dental model to match the movement speed of a reference dental model clip. In some cases, the generating comprises aligning the display of at least part of the provided dental model with a reference dental model clip. In some cases, the generating comprises changing the display perspective of at least part of the provided dental model to match the display perspective of a reference dental model clip. In some cases, the generating comprises adding transparent or colorful effects to at least part of the provided dental model.

In some cases, the dental model clip is a movie, video, or animation. In some cases, the dental model clip is less than 1 minute long. In some cases, the dental model clip is about 0.25 minutes, about 0.5 minutes, about 1 minute, about 1.5 minutes, or about 2 minutes. In some cases, the dental model clip is at least about 0.25 minutes, about 0.5 minutes, about 1 minute, or about 1.5 minutes. In some cases, the dental model clip is at most about 0.5 minutes, about 1 minute, about 1.5 minutes, or about 2 minutes.

In some cases, the method further comprises presenting the dental model clip simultaneously with a reference dental scan clip. In some cases, the method further comprises underlying or overlying the dental model clip with a reference dental scan clip. In some cases, differences between the dental model clip and the reference dental scan clip are marked.

The differences between the dental model clip and the reference clip may be used to evaluate or monitor the progress of a dental condition or orthodontia treatment plan for a patient. The dental condition may comprise (i) a movement of one or more teeth of the subject, (ii) an accumulation of plaque on the one or more teeth of the subject, (iii) a change in a color or a structure of the one or more teeth of the subject, (iv) a change in a color or a structure of a tissue adjacent to the one or more teeth of the subject, and/or (v) a presence or lack of presence of one or more cavities. In some cases, the differences between the dental model clip and the reference clip may be used to assess the subject's actual progress in relation to a dental treatment plan based at least in part on a comparison of (i) the one or more changes in the dental structure or the dental condition of the subject and (ii) a planned or estimated change in the dental structure or the dental condition of the subject.

### Remote Dental Monitoring

In one aspect, the present disclosure provides a computer-implemented method for remote dental monitoring, the method comprising: providing a dental scan clip of a user; providing a reference dental scan clip of the user or a dental model clip of the user; providing a presentation device; and presenting the clips simultaneously.

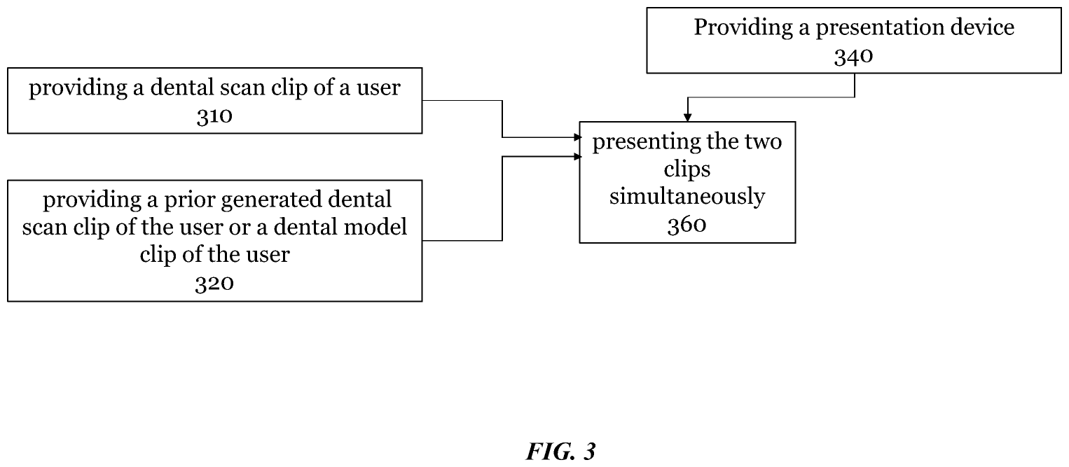

FIG. 3 is a flow-chart schematically illustrating a method for remote dental monitoring, in accordance with some embodiments. As shown, the method comprises providing a dental scan clip of a user 310; providing a reference dental scan clip of the user or a dental model clip of the user 320; providing a presentation device 340; and presenting the clips simultaneously 360.

As used herein, remote monitoring and dental imaging may refer to monitoring a dental anatomy or a dental condition of a patient and taking images of the dental anatomy at one or more locations remote from the patient or dentist. For example, a dentist or a medical specialist may monitor the dental anatomy or dental condition in a first location that is different than a second location where the patient is located. The first location and the second location may be separated by a distance spanning at least 1 meter, 1 kilometer, 10 kilometers, 100 kilometers, 1000 kilometers, or more. The remote monitoring may be performed by assessing a dental anatomy or a dental condition of the subject using one or more intraoral images captured by the subject when the patient is located remotely from the dentist or a dental office. In some cases, the remote monitoring may be performed in real-time such that a dentist is able to assess the dental anatomy or the dental condition when a subject uses a mobile device to acquire one or more intraoral images of one or more intraoral regions in the patient's mouth. The remote monitoring and dental imaging may be performed using equipment, hardware, and/or software that is not physically located at a dental office.

### Computer System

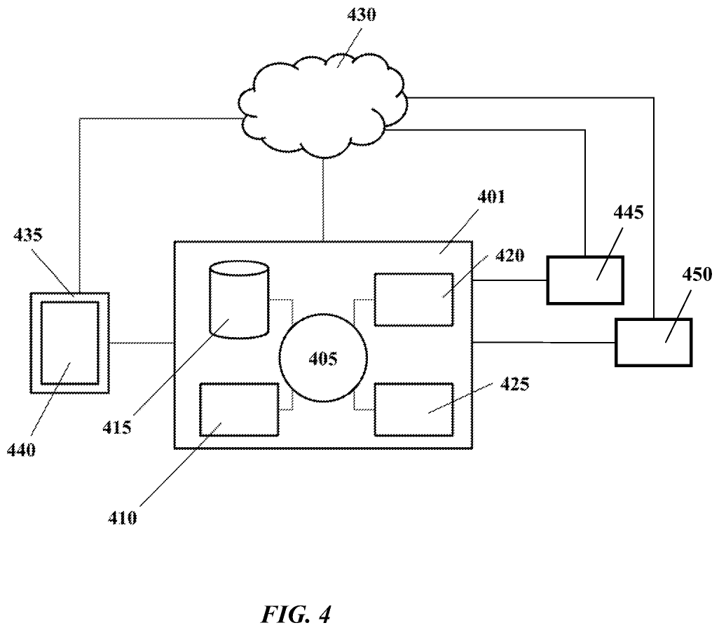

FIG. 4 shows a computer system 401 that is programmed or otherwise configured to implement a method for dental assessment and editing or generating dental scan clip or dental model clip. The computer system 401 may be configured to, for example, process intraoral videos or images captured using the camera of the mobile device, and determine at least one of the dental assessment properties during the dental assessment. The computer system 401 can be an electronic device of a user or a computer system that is remotely located with respect to the electronic device. The electronic device can be a mobile electronic device. The computer system 401 can be a smartphone.

The computer system 401 may include a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk, Solid State drive or equivalent storge unit), communication interface 420 (e.g., network adaptor) for communicating with one or more other systems, and peripheral devices 425, such as cache, other memory, data storage and/or electronic display adaptors. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 430 can include one or more computer servers, which can enable distributed computing, such as cloud computing. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are located external to the computer system 401 (e.g., on a remote server that is in communication with the computer system 401 through an intranet or the Internet).

The computer system 401 can communicate with one or more remote computer systems through the network 430. For instance, the computer system 401 can communicate with a remote computer system of a user (e.g., a subject, a dental patient, or a dentist). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a storage unit. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440 for providing, for example, a portal for a subject or a dental patient to view one or more intraoral images or videos captured using a mobile device of the subject or the dental patient. In some cases, the electronic display 435 may be the feedback element providing the generated output, for example displaying message or shape or light in accordance to some embodiments. The portal may be provided through an application programming interface (API). A user or entity can also interact with various elements in the portal via the UI. Examples of UI's include, without limitation, a graphical user interface (GUI) and web-based user interface.

The computer system 401 can include or be in communication with a Camera 445 for providing, for example, ability to capture videos or images of the user or a dental patient. And for example, retrieve at least one dental scan date (such as optical object distance) that can be used to generate a dental scan clip or dental model clip.

Methods and systems of the present disclosure can be implemented by way of one or more algorithms. An algorithm can be implemented by way of software upon execution by the central processing unit 405. The algorithm can, for example, implement a method for dental scan. The method may comprise processing videos or images captured using the camera of the mobile device or processing dental assessment data sensed by at least one sensor that can be used to analyze and compare to at least one dental assessment properties and executed to generate output.

While embodiments have been shown and described herein, it will be obvious to those skilled in the art that such embodiments are provided by way of example only. It is not intended that the systems and methods described herein be limited by the specific examples provided within the specification. While the systems and methods described herein has been described with reference to the aforementioned specification, the descriptions and illustrations of the embodiments herein are not meant to be construed in a limiting sense.

Numerous variations, changes, and substitutions will now occur to those skilled in the art without departing from the systems and methods described herein. Furthermore, it shall be understood that all aspects of the systems and methods described herein are not limited to the specific depictions, configurations or relative proportions set forth herein which depend upon a variety of conditions and variables. It should be understood that various alternatives to the embodiments described herein may be employed in practicing the systems and methods described herein. It is therefore contemplated that the systems and methods described herein shall also cover any such alternatives, modifications, variations or equivalents. It is intended that the following claims define the scope of the systems and methods described herein and that methods and structures within the scope of these claims and their equivalents be covered thereby.

1. A method to generate a dental scan clip, comprising:
(a) providing a clip start point, a clip end point, and a dental scan parameter; and (b) editing a dental scan in accordance with the clip start point, the clip end point, and the dental scan parameter to generate the dental scan clip.

2. The method of claim 1, wherein the clip start point is determined by a point in the dental scan at which a dental landmark is identified within the dental scan or wherein the clip end point is determined by a point in the dental scan at which a dental landmark is no longer visible in the dental scan.

3. The method of claim 2, wherein the dental landmark is a maxillary tuberosity, a retromolar pad, or a tooth.

4. The method according to claim 1, wherein the dental scan parameter comprises ensuring an upper teeth arch, a lower teeth arch, or a bite arch remains horizontal in the dental scan frame.

5. The method according to claim 1, wherein the dental scan parameter comprises ensuring one or more spaces between two or more teeth remains vertical in the dental scan frame.

6. The method according to claim 1, wherein the dental scan parameter comprises ensuring an imaginary line between two dental landmarks remains horizontal in the dental scan frame.

7. The method according to claim 1, wherein the dental scan parameter comprises ensuring the movement speed of a dental scan remains constant.

8. The method according to claim 1, wherein the dental scan provided is comprises one or more videos or images.

9. The method according to claim 1, wherein the dental scan provided is a video or image acquired with a mobile device.

10. The method according to claim 1, wherein the editing comprises matching at least part of the provided dental scan parameters to a reference dental scan clip.

11. The method according to claim 1, wherein the dental scan clip is a movie, video, or animation.

12. The method according to claim 1, wherein the dental scan clip is less than 1 minute long.

13. The method according to claim 1, further comprising presenting the dental scan clip simultaneously with a reference dental scan clip.

14. A system to generate a dental scan clip, in accordance with claim 1.

15. A method to transform three dimensional (3D) dental data into a dental model clip, comprising:
(a) providing the 3D dental data;
(b) providing a graphic visualization tool;
(c) using the graphic visualization tool to generate a dental model from the 3D dental data;
(d) providing a clip start point, a clip end point, and a dental model parameter; and
(e) generating the dental model clip from the dental model in accordance with the clip start point, the clip end point, and the dental model parameter.

16. The method according to claim 15, further comprising presenting the dental model clip simultaneously with a reference dental scan clip.

17. The method according to claim 15, further comprising underlying or overlying the dental model clip with a reference dental scan clip.

18. The method according to claim 17, wherein differences between the dental model clip and the reference dental scan clip are marked.

19. A computer-implemented method for remote dental monitoring, the method comprising:
providing a dental scan clip of a user; providing a reference dental scan clip of the user or a dental model clip of the user; and simultaneously presenting the dental scan clip with the reference dental scan clip or the dental model clip on a graphical display.
